(19) United States
(12) Patent Application Publication
(10) Pub. No.: US 2020/0126651 A1
(43) Pub. Date: Apr. 23, 2020

(54) Systems and Methods for a Personal Healthcare Manager

(71) Applicant: LifeTime App, Inc., San Jose, CA (US)

(72) Inventor: Igor Nikolayevich Hismatullin, San Jose, CA (US)

(73) Assignee: LifeTime App, Inc., San Jose, CA (US)

(21) Appl. No.: 16/659,135

(22) Filed: Oct. 21, 2019

Related U.S. Application Data
(60) Provisional application No. 62/748,155, filed on Oct. 19, 2018.

(51) Int. Cl. G16H 20/00; G16H 50/20; G16H 50/70; G16H 10/60; G16H 80/00

(52) U.S. Cl. CPC G16H 20/00; G16H 50/20; G16H 80/00; G16H 10/60; G16H 50/70

(57) ABSTRACT

Systems, methods, and computing devices with novel functionalities that improve the coordination of and communication between multiple electronic devices to treat individuals who are ill, constantly monitor their health, prevent them from getting sick, and optimize their health for longevity. The disclosed technology empowers patients to take control of their personal health and enhances the interaction between patients, practitioners, third-party services, and healthcare systems to provide effective and efficient professional healthcare to the patient.

### CROSS-REFERENCE TO RELATED APPLICATIONS

This application claims benefit to U.S. Provisional Application No. 62/748,155, filed on Oct. 19, 2018, entitled “System and Method for an Action Health Monitoring System”, the contents of which are incorporated by reference herein as though set forth in their entirety, and to which priority and benefit are claimed.

### TECHNICAL FIELD

The present disclosure relates generally to systems and methods for patient healthcare management. More particularly, the present disclosure relates to systems, devices, and methods for assisting medical practitioners and patients in the personal management of the patient's healthcare.

### BACKGROUND INFORMATION

Life expectancy for the general population has increased dramatically over the last century due to advancements, for example, in areas such as sanitation, food production, and medicine. Similarly, recent innovation in technology is enabling individuals to obtain access to information and resources that can dramatically improve their health and increase their longevity. Yet many aspects of the healthcare profession—such as patient care and the working practices of healthcare professionals—have been somewhat slow in the adaption of new technology. For example, three weeks after making an appointment to address a non-urgent symptom, an individual may finally visit with her primary care physician for a total of only five minutes, wherein the doctor will review the information provided by the patient via the nurse, briefly and hastily discuss all potential diagnosis and options for treatment, and instruct the patient to use any prescribed medication per the pharmacist's instructions. The impersonal and disconnected relationships between medical practitioners and patients result in delayed diagnosis, improper treatment, increased healthcare costs, prolonged illness, and frustration for all involved.

Thus, what is needed are new and improved technologies that improve the interaction between patients, practitioners, and healthcare systems to provide immediate and professional healthcare to the patient—leading to better diagnoses, tailored treatment options, implemented preventative measures, reduced costs, improved relationships between medical practitioners and their patients, and the overall enhancement of the practice of medicine.

### SUMMARY OF THE DISCLOSURE

The following presents a simplified overview of the example embodiments in order to provide a basic understanding of some embodiments of the present disclosure. This overview is not an extensive overview of the example embodiments. It is intended to neither identify key or critical elements of the example embodiments nor delineate the scope of the appended claims. Its sole purpose is to present some concepts of the example embodiments in a simplified form as a prelude to the more detailed description that is presented herein below. It is to be understood that both the following general description and the following detailed description are exemplary and explanatory only and are not restrictive.

The present disclosure is directed to computer devices, systems, and methods for receiving, by a system, electronic medical data of a first user; comparing, by the system, the electronic medical data to a plurality of healthcare data in one or more databases; identifying, by the system, at least one medical consideration in the electronic medical data upon the comparison of the electronic medical data to the plurality of healthcare data; comparing, by the system, the identified medical consideration to a plurality of medical consideration data in one or more databases, wherein the plurality of medical consideration data comprises one or more medical considerations and one or more action items addressing the one or more medical considerations; identifying, by the system, at least one action item addressing the identified medical consideration; generating, by the system, at least one functional electronic action item corresponding with the identified action item, wherein the functional electronic action item comprises a required data input functionality; and transmitting, by the system, the functional electronic action item to one or more servers.

Still other advantages, embodiments, and features of the subject disclosure will become readily apparent to those of ordinary skill in the art from the following description wherein there is shown and described a preferred embodiment of the present disclosure, simply by way of illustration of one of the best modes best suited to carry out the subject disclosure. As will be realized, the present disclosure is capable of other different embodiments and its several details are capable of modifications in various obvious embodiments all without departing from, or limiting, the scope herein. Accordingly, the drawings and descriptions will be regarded as illustrative in nature and not as restrictive.

### BRIEF DESCRIPTION OF THE DRAWINGS

The accompanying drawings, which are incorporated in and constitute a part of this specification, illustrate embodiments of the disclosure and together with the general description of the disclosure given above and the detailed description of the drawings given below, serve to explain the principles of the disclosure. In certain instances, details that are not necessary for an understanding of the disclosure or that render other details difficult to perceive may have been omitted.

FIG. 1A illustrates an example overview of one or more implementations described herein.

FIG. 1B illustrates an example overview of the functionalities of a personal health manager system for one or more implementations described herein.

FIG. 2A illustrates example functional components of a personal health manager system, in accordance with certain implementations described herein.

FIG. 2B illustrates example functional components of a personal health manager system, in accordance with certain implementations described herein.

FIG. 3 illustrates example processes for the generation, organization, and distribution of action cards by a personal health manager system, in accordance with certain implementations described herein.

FIG. 4A conceptually illustrates an example of a patient interface rendered by a personal health manager system, in accordance with certain implementations described herein.

FIG. 4B conceptually illustrates an example of an action card rendered by a personal health manager system, in accordance with certain implementations described herein.

FIG. 4C conceptually illustrates an example of a To-Do List rendered by a personal health manager system, in accordance with certain implementations described herein.

FIG. 5 illustrates an example flowchart for the creation of To-Do Items by a personal health manager system, in accordance with certain implementations described herein.

FIG. 6A illustrates example processes for the execution of a To-Do Item by a personal health manager system, in accordance with certain implementations described herein.

FIG. 6B illustrates example processes for the execution of a To-Do Item by a personal health manager system, in accordance with certain implementations described herein.

FIG. 7 is a functional block diagram generally illustrating an embodiment of a network system for a personal health manager system.

FIG. 8 is a functional block diagram generally illustrating an embodiment of an electronic device system for a personal health manager system.

### DETAILED DESCRIPTION OF EMBODIMENTS

Before the present systems and methods are disclosed and described, it is to be understood that the systems and methods are not limited to specific methods, specific components, or to particular implementations. It is also to be understood that the terminology used herein is for the purpose of describing particular embodiments only and is not intended to be limiting. Various embodiments are described with reference to the drawings. In the following description, for purposes of explanation, numerous specific details are set forth in order to provide a thorough understanding of one or more embodiments. It may be evident, however, that the various embodiments may be practiced without these specific details. In other instances, well-known structures and devices are shown in block diagram form to facilitate describing these embodiments.

The current innovation and progress in technology is felt by many. For example, the majority of the U.S. population has some version of a smart electronic device (a device with communication functionalities, a high-processing system, and an operating system that can carry out a variety of actions). Similarly, the computing power of computer systems today is enabling the compilation and analysis of vast amounts of data, which computer systems can learn from to begin carrying out specific tasks and make inferences in a process known as machine learning. Improving on this technology, the computing devices, systems, and methods described herein disclose a technology with novel functionalities that improve the coordination of and communication between multiple electronic devices to treat individuals who are ill, constantly monitor their health, prevent them from getting sick, and optimize their health for longevity. In so doing, the disclosed technology empowers patients to take control of their personal health and enhances the interaction between patients, practitioners, and healthcare systems to provide effective and efficient professional healthcare to the patient.

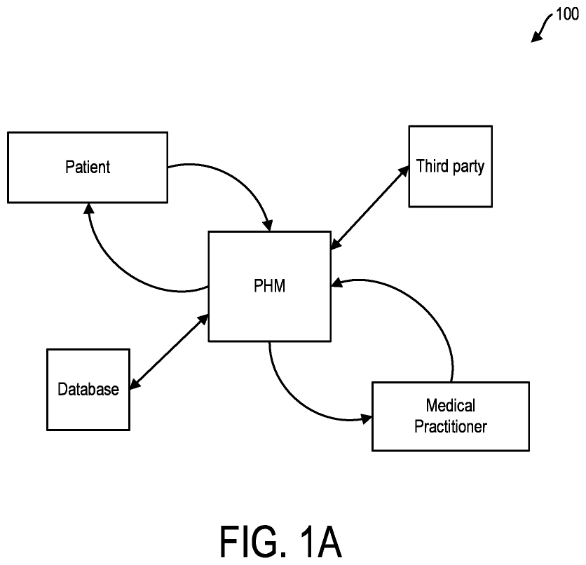

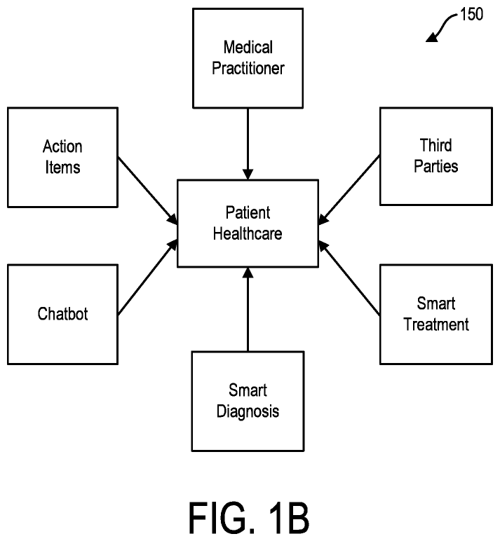

FIG. 1A illustrates an example overview 100 of one or more implementations described herein. As shown in FIG. 1A, an example overview 100 comprises a personal health manager system (“PHM”) that may coordinate and enhance the interactions between patients, medical practitioners, and third parties, while also searching for, receiving, and utilizing data received from internal and external databases.

In an illustrative embodiment of the example overview 100, the PHM may receive medical data provided by the patient (such as a description of existing symptoms or a self-diagnosis of vital signs) and perform an internal analysis and evaluation to generate personalized medical considerations and corresponding treatment plans. Medical considerations may be defined as the determination of a factor relevant to an individual's current or future health. Afterwards, the PHM may transfer the generated medical consideration and treatment proposal to a medical practitioner. Following electronic confirmation of review by the medical practitioner, the PHM may receive data from the medical practitioner that amends or replaces the generated medical considerations/treatment plans. The PHM may then perform additional analysis and evaluation and generate a new medical consideration and treatment, which is rendered on the patient's or the medical practitioner's electronic device. While the PHM is performing these actions, it may also be sending the patient's data to third parties, from which it will receive information regarding third-parties' products or services that are relevant/related to any of the generated medical considerations/treatment options. Concurrently, the PHM may search its internal database to determine whether it has previously received similar data from the patient and what occurred following the receipt of that data. And the PHM may search an external pharmacy database to determine medication that is prohibited based on the patient's inputted data. As a result, the PHM may carry out technical functionalities—previously unavailable—that: (i) streamline the medical practitioner's workflow and assist in delivering primary care to the patient; (ii) when combined, serve as a dynamic and operational healthcare management tool for the patient; and (iii) enable third parties, previously exempt from the doctor-patient relationship, to communicate and coordinate with the patient and/or medical practitioner for the purpose of enhancing the medical care of the patient. Additionally, the PHM may employ artificial intelligence to analyze and detect healthcare issues or medical considerations before the patient is aware. In doing so, the PHM may suggest lifestyle modifications, intervene and deliver content to the patient to prevent illness, optimize health, and/or communicate with a provider.

For the purposes of this disclosure, a patient may be defined as any individual that accesses the PHM for the purpose of improving his/her health. For example, a patient may be an individual that has never consulted with a medical practitioner, such as a doctor, but seeks health advice because he is feeling ill or is overweight. Or, a patient may be an individual with a diagnosed illness that is seeking treatment or a second opinion.

For the purposes of this disclosure, a medical practitioner may be defined as any individual or entity that can provide health management. For example, a nurse or a doctor may be a medical practitioner. Or, an urgent care clinic may act as a medical practitioner.

For the purposes of this disclosure, a third party may be defined as any individual, organization, or entity that is neither the patient nor the medical practitioner providing medical care. For example, a third party may be a separate medical practitioner, such as a healthcare system or nurse. Or a third party may be a pharmacy or pharmacist. Another example may be a company providing its products/services that are relevant to either the health management of the patient or the medical care provided by the medical practitioner.

For the purposes of this disclosure, a database may be defined as a structured set of data that is electronically stored, such as in a computer or server. A database may be internal or external in relation to the PHM. For example, a database may be the medical practitioner's medical history of the patient that resides within a database in the PHM. Or, a database may be a company that provides electronic health records, such as drchrono™. In another example, a database may be any set of data that makes up a health information exchange. A database can also be any compilation of current state or federal law relevant to the medical field or practice of medicine.

For the purposes of this disclosure, electronic medical data may be defined as electronic information relevant to the health of an individual. Other terms for electronic medical data include healthcare data. Examples of electronic medical data specific to the patient that the PHM may use include: Conditions (current and past): chronic and acute conditions (ICD-10); physical exam data (including vital signs, physical assessment notes); test results (laboratory data, diagnostic imaging data, pathology results, specialized testings); information related to the patient's allergies; surgical history; medication history; vaccination history; lifestyle history (including sexual history, tobacco use, drug use, alcohol use, exercise habits, dietary habits, sleep habits, sport/activity habits, and activities); social history (close relationships, occupations, community support); mental-health history; genetic information; general medical history; family history; demographics (race/ethnicity, country of origin, age, sex, occupation); geographical information (Including zip codes, county, country, address); and medical encounters (hospital admissions, consultations, routine checkups).

FIG. 1B illustrates an example overview 150 of the functionalities of a personal health manager system for one or more implementations described herein. As shown in FIG. 1B, an example overview 150 may comprise the following functionalities: improved coordination and communication capabilities with medical practitioners or third parties; internal machine learning components such as a smart diagnosis component, smart risk analysis, and smart treatment component; use of communication capabilities such as an instant messaging chatbot; and the novel generation of functional action items, such as personalized action cards.

For purposes of this disclosure, a smart diagnosis component (“Diagnosis Engine”) may be defined as an electronic system that uses logic to gather data relevant to a patient and diagnose for any medical conditions. For example, the Diagnosis Engine may aggregate patient data, internal data, and data from various sources external to the PHM and flag issues detected. Following any detection or diagnosis of health issues, the PHM, via the Diagnosis Engine, may generate a summary of the diagnosis and render it to a medical practitioner or the patient. The Diagnosis Engine may be a stand-alone system independent from other components of the PHM or it may be a sub-component of other components or systems of the PHM.

For purposes of this disclosure, a smart risk analysis component (“Risk Engine”) may be defined as an electronic system that uses logic to gather data relevant to a patient and analyze for the potential risk of any current or future medical conditions. For example, the Risk Engine may track single-point data overtime and analyze for the risk of a medical condition. Similarly, it may continuously update medical calculators within the PHM to determine the potential risk of a condition developing. Following any detection or diagnosis of the potential risk of health issues, the PHM, via the Risk Engine, may generate a summary of the analysis and render it to a medical practitioner or patient. The Risk Engine may be functioning at all times to determine the potential risk of medical considerations or conditions, regardless of whether a patient or medical practitioner is inputting new data. The Risk Engine may be a stand-alone system independent from other components of the PHM or it may be a sub-component of other components or systems of the PHM.

For purposes of this disclosure, a smart treatment component (“Treatment Engine”) may be defined as an electronic system that uses logic to gather and analyze data, such as data from the Diagnosis Engine or Risk Engine, to generate one or more medical treatment options. It may generate a summary of the analysis and render it to a medical practitioner and/or the patient. The Treatment Engine may be a stand-alone system independent from other components of the PHM or it may be a sub-component of other components or systems of the PHM.

For purposes of this disclosure, communication capabilities may be defined as technology that enables communication between the patient and medical practitioner. An example may be an instant messaging chatbot that allows for immediate communication. Communication functionalities may also include teleconferencing, document sharing, and data retention components. Communication functionalities may coordinate with the Diagnosis Engine, Risk Engine, and Treatment Engine. For example, the Diagnosis Engine may determine what data is needed to generate a diagnosis and request this information from the patient through the chatbot. Communication capabilities, such as chatbots, provided the additional benefit of storing, organizing, and indexing communication history—resulting in additional data that may be used by the Risk Engine or by the PHM in general to further generate to-do items, action cards, diagnoses, and treatment options. Patients and medical practitioners, along with third parties, may also have access to the communication history, thereby reducing duplicative actions, loss of information, and an easily accessible source of personal healthcare information.

For purposes of this disclosure, a functional, electronic item (such as an action card) may be defined as an electronic, functional rendering of a proposed course of action the patient should take. The proposed course of action is determined by the PHM, stemming from its Diagnosis, Risk, and Treatment Engines. Novel aspects of it comprise the capacity for a user to engage and interact with a functional, electronic course of action—which may comprise a required data-input functionality—that is tailored to address the preventative, diagnosis, risk, and treatment aspects specific to the user's health.

Examples of courses of action that can be tailored to the specific profile of the patient are as follows: instructing the patient to watch a video or read an article, wherein the patient may afterwards be quizzed on the content of the video or article; requesting the input of specific data for the PHM to better understand the patient's general behavior, personal history, medical history, or preferences (these cards help the system learn about the patient, serve more relevant cards, assist in suggested diagnosis, risk assessments, treatment plans, and lifetime projections); requesting the input of specific, quantifiable data for the PHM to better assess a user's specific behavior (example: How many hours of sleep did you get last night or what is your blood pressure today?); actions related to personal goals set by the patient, wherein the cards may be linked to third-party apps or products; recommending a product or service to the patient for purchase; notice to schedule an appointment; or recommendation that the patient complete a task.

In one embodiment, the PHM may receive data obtained through the chatbot that is related to only a patient's dietary and sleeping habits, upon which it may analyze this through its Diagnosis, Risk, and Treatment Engines to determine the patient's remaining life expectancy. Based on the received data and determined life expectancy, the PHM may generate action cards that serve to increase the patient's life expectancy and/or request additional information that may produce further data the PHM may then use to re-analyze the patient. Examples of action cards are as follows: confirmation that one has consumed the recommended serving of fruit and vegetables, data input of the frequency one smokes cigarettes, answering questions based on the review of a published article describing benefits of exercise, and data input of a blood pressure screening.

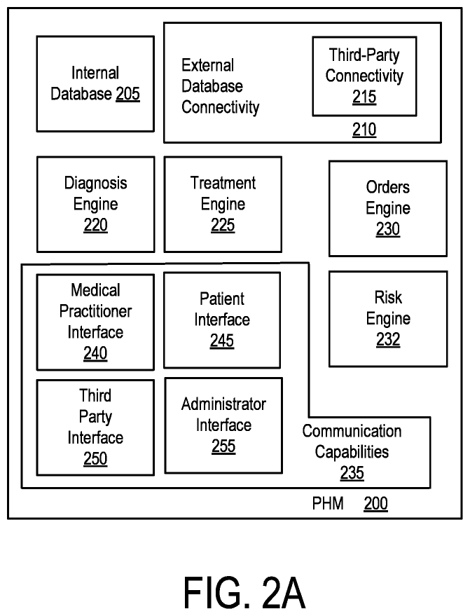

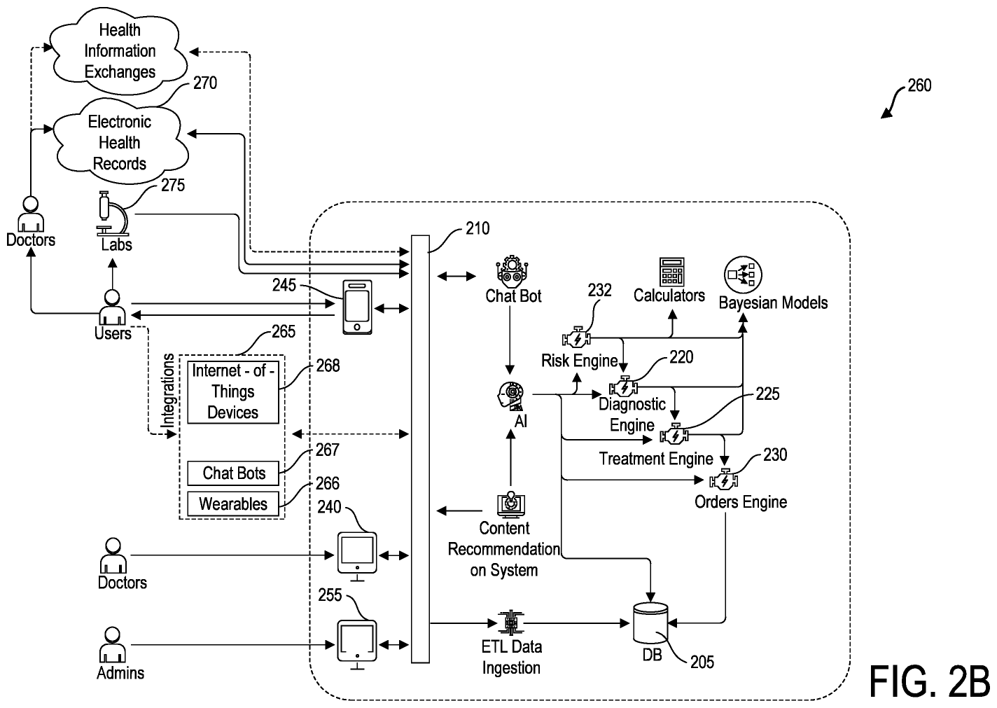

FIG. 2A illustrates example functional components of a personal health manager system 200, in accordance with certain implementations described herein. As shown in FIG. 2A, the PHM system 200 may comprise one or more of the following functional components: an internal database 205, external database connectivity 210, a Diagnostic Engine 220, a Treatment Engine 225, an Orders Engine 230, a Risk Engine 232, and a Communication Capabilities 235 component.

An internal database 205 may be a database that resides within the PHM system 200. The internal database 205 may be necessary or helpful in carrying out the functionalities of the Diagnosis Engine, Risk Engine, Treatment Engine, or other components of the PHM system 200. For example, the internal database 205 may include data such as the chatbot history between patients and medical practitioners, previously generated action cards, risk assessments, or diagnosis and treatment options previously generated by the PHM system 200.

External database connectivity 210 may be the connection functionalities the PHM system 200 uses to transmit data to and receive data from users, medical practitioners, or any database that is external to the PHM system 200. Examples of data received via the external database connectivity 210 include: receiving data from Internet-of-things devices such as consumer-connected devices, industrial, and enterprise Internet-of-things devices; receiving electronic health records from a third party; receiving relevant statutory laws from a government entity; receiving data for relevant medical products that can be purchased, such as a wrist brace; and receiving data for relevant medical services that can be ordered, such as lab testing for an infectious disease. Examples of transmitting data via the external database connectivity 210 include transmitting notice of a required surgery to a surgeon's office as a referral or transmitting notice that a patient may benefit from the use a wheelchair to a wheelchair provider. External database connectivity 210 may include third-party connectivity 215, which may be the connection functionalities the PHM system 200 uses to connect with third parties.

Similar to the Diagnosis Engine 220 or the Treatment Engine 225, the Orders Engine 230 is an electronic system that uses logic to gather and analyze data, such as data received from a medical practitioner or a chatbot, to then execute to-do items. To-do items may be defined as a process created by the PHM system 200 that requires execution and completion, such as transmitting treatment data to a patient, requesting a medical device, and requesting additional information from the patient or medical practitioner after diagnosis of a disease. The Orders Engine 230 may generate a summary of the analysis and provide it to the patient, medical practitioner, or third-party. The Orders Engine 230 may be a stand-alone system independent from other components of the PHM or it may be a sub-component of other components or systems of the PHM.

The communication capabilities 235 enables communication between the patient, medical practitioner, and third parties. In addition to technology such as the chatbot, communication capabilities 235 may also comprise a medical practitioner interface 240, a patient interface 245, a third-party interface 250, or an administrator interface 255. These interfaces may include communication capabilities such as the transmission of data, messaging features such as chatbot, and a dashboard component that enables the patient, practitioner, or third party to evaluate its relationship and administrative settings with the PHM.

FIG. 2B illustrates example functional components of a personal health manager system, in accordance with certain implementations described herein. FIG. 2B discloses an embodiment 260 of the functional components of a personal health manager system. For example, the PHM system 260 may comprise one or more of the following functional components: an internal database 205, external database connectivity 210, a Diagnostic Engine 220, a Treatment Engine 225, an Orders Engine 230, a Risk Engine 232, and an artificial intelligence 280 component.

External database connectivity 210 may be the connection functionalities the PHM system 260 uses to transmit data to and receive data from users, medical practitioners, or any database that is external to the PHM system 260. Examples of data received via the external database connectivity 210 include: receiving data from integrated devices 265 such as consumer-connected devices 266, chatbots 267, and industrial and enterprise Internet-of-things devices 268; receiving electronic health records 270 from a third party; receiving relevant statutory laws from a government entity; receiving data for relevant medical products that can be purchased, such as a wrist brace; and receiving data for relevant medical services that can be ordered 275, such as lab testing for an infectious disease.

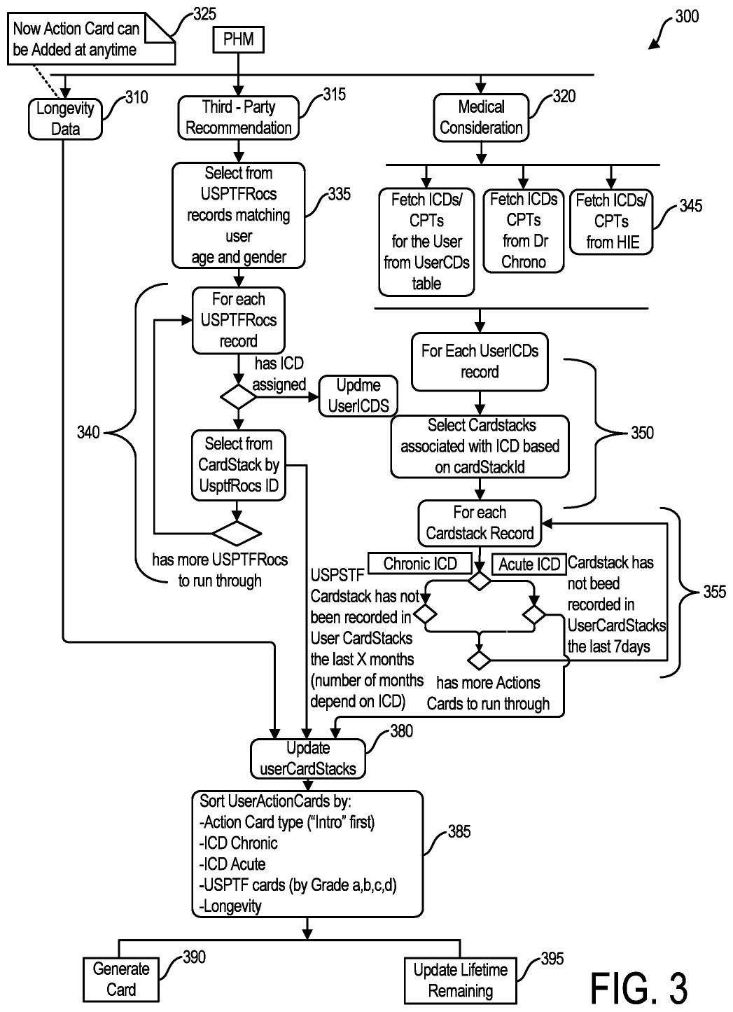

FIG. 3 illustrates example processes 300 for the generation, organization, and distribution of action cards by a Personal Health Manager system, in accordance with certain implementations described herein. The PHM accomplishes this through the Diagnosis, Risk, and Treatment Engines. As shown in FIG. 3, the PHM may generate and/or update 390 (collectively, “generate”) an action card via four processes. However, the PHM may generate 390 action cards through other processes not disclosed in FIG. 3. For example, the action cards may be generated by and organized on the basis of: type (i.e. “Intro cards” first), chronic diagnosis, acute diagnosis, third-party cards (i.e. cards based on data received from the U.S. Preventive Services Task Force), actions to increase longevity, and immediate/urgent actions. Additionally, the PHM may have a pre-determined order for the distribution of cards to individual patients and may alter this order based on new data received and/or new cards generated.

In one embodiment, the PHM may generate an action card based on the process of determining a patient's longevity. It may do so by generating a longevity card based on data related to the longevity of the patient's health 310. The longevity card is a functional action card with data that the patient may view and act upon, such as by inputting data into the longevity card. However, the PHM may select generated longevity cards not already in the patient's set of generated cards or the PHM may accept pre-generated cards 325 and add them to the database of longevity cards. Once the PHM has generated or received longevity data 310, it may update the pre-determined set 380 of action cards for the patient and organize the generated longevity card according to its type. The PHM may then generate 390 the card on the patient interface and/or update 395 the patient's remaining lifetime based on the longevity data 310. Similarly, the PHM may generate an action card based on the process of optimizing a patient's health. For example, the PHM may optimize a patient's health by determining preventative steps a patient can take that go beyond standard preventative measures and generate actions cards that the patient can engage with to improve and optimize her health.

In another embodiment, the PHM may generate an action card through the process of receiving relevant data from a third party 315. For example, the PHM may receive data from the U.S. Preventive Services Task Force (“USPSTF”), an organization of experts in primary care and prevention that systematically review the evidence of effectiveness and develop recommendations for clinical preventive services. The PHM may select from a USPSTF database records that match the patient's age and gender. For each record received, the PHM may go through a sub-process 340 that updates the patient's diagnosis and treatment evaluations and receives, as needed, additional records from the USPSTF. Once the PHM has generated or updated action cards based on third-party data 315, it may update the pre-determined set 380 of action cards for the patient and organize the generated card according to its type. The PHM may then generate 390 the card on the patient interface and/or update 395 the patient's remaining lifetime based on the data inherent in the generated longevity card 310.

In a separate embodiment, the PHM may generate an action card with the intent of diagnosing or assessing the potential medical risks and considerations 320 of the patient. To accomplish this, the PHM may fetch 345 data from sources such as from the user, from a third party like drchrono™, or from health information exchange processes. After receiving the data, the PHM may correspond 350 the data to other data related to medical risks and considerations already received or generated. In addition, for each piece of data received, the PHM may go through a sub-process 355 that further screens the data and updates the patient's diagnosis, risks, longevity, optimization, and treatment considerations as needed. Once the PHM has gone through this process, it may update the pre-determined set 380 of action cards for the patient and organize the generated card according to its type. The PHM may then generate 390 the card on the patient interface and/or update 395 the patient's remaining lifetime based on the data inherent in the generated diagnosis card.

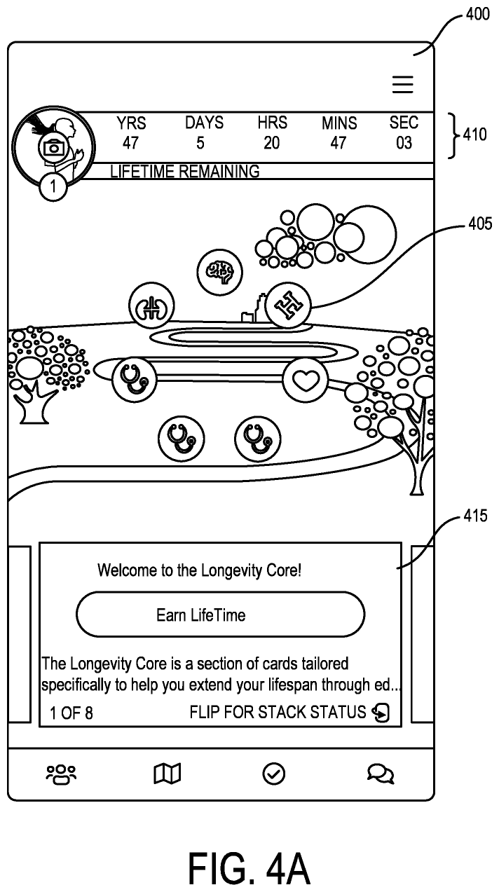

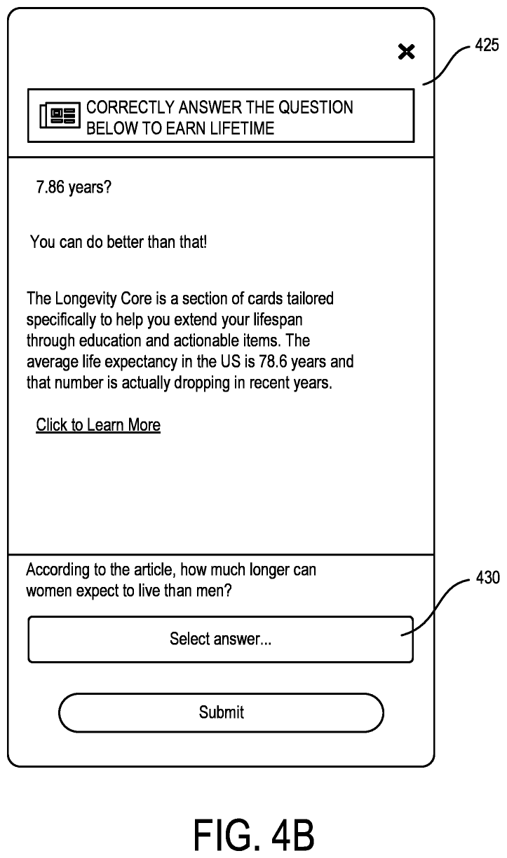

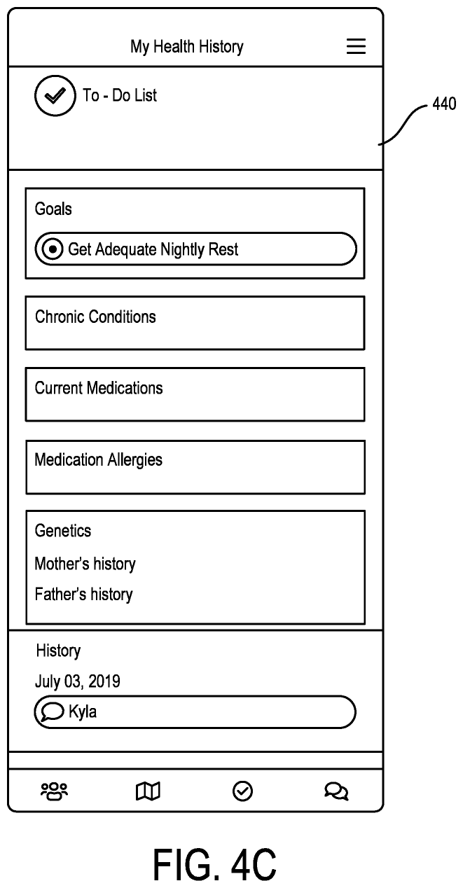

FIG. 4A conceptually illustrates an example of a patient interface 400 rendered by a Personal Health Manager system, in accordance with certain implementations described herein. As shown in FIG. 4A, a patient interface rendered by the PHM may have functional tools 405 that enable the patient to navigate the PHM. Examples of functional tools include connection links to: a list of individuals related to the patient; the main page of the interface; history and to-do list items, and communication features such as a chatbot. The patient interface 400 may display the patient's remaining lifetime 410, organize to-do cards, and display individual To-Do Cards 415.

FIG. 4B conceptually illustrates one example of an action card 425 rendered by a Personal Health Manager system, in accordance with certain implementations described herein. FIG. 4B displays an action that requires the patient to review an article. The PHM has generated the specific article and corresponding article based on the previously generated diagnosis, risk analysis, and/or treatment of the patient. In other words, the action 430 is personally tailored to address the patient's diagnosis, risk analysis, or treatment options. The action card 425 is functional and may receive data from the patient, which it will incorporate into future diagnosis, risk analysis, and treatment processes.

FIG. 4C conceptually illustrates an example of a To-Do List 440 rendered by a Personal Health Manager system, in accordance with certain implementations described herein. As shown in FIG. 4C, the PHM may generate a functional To-Do List 440 for the management and organization of the patient's personal data. The To-Do List 440 may provide an overview of to-do items the PHM has generated, diagnosis, risk analysis, and treatment options generated by the PHM, and communication history, such as chatbot history. It may also serve as a repository of relevant healthcare/medical data, such as chronic conditions, current medications, medication allergies, and genetic history of the patient. As the patient engages and interacts with the PHM over a prolonged period of time, the PHM may store this data in its internal database and display it on the patient interface 400 in the form of a To-Do List 440.

Similar to the disclosures of FIGS. 4A-4C, the PHM may render and display interfaces for medical practitioners, third-parties (such as companies advertising their services/products), and administrative entities. These interfaces enable the respective entities to engage and interact with the PHM, wherein they may input, transmit, and receive data. Additionally, the PHM may communicate with the servers or databases of these entities, wherein the dynamic relationship between the PHM and these entities is configured into a functional interface.

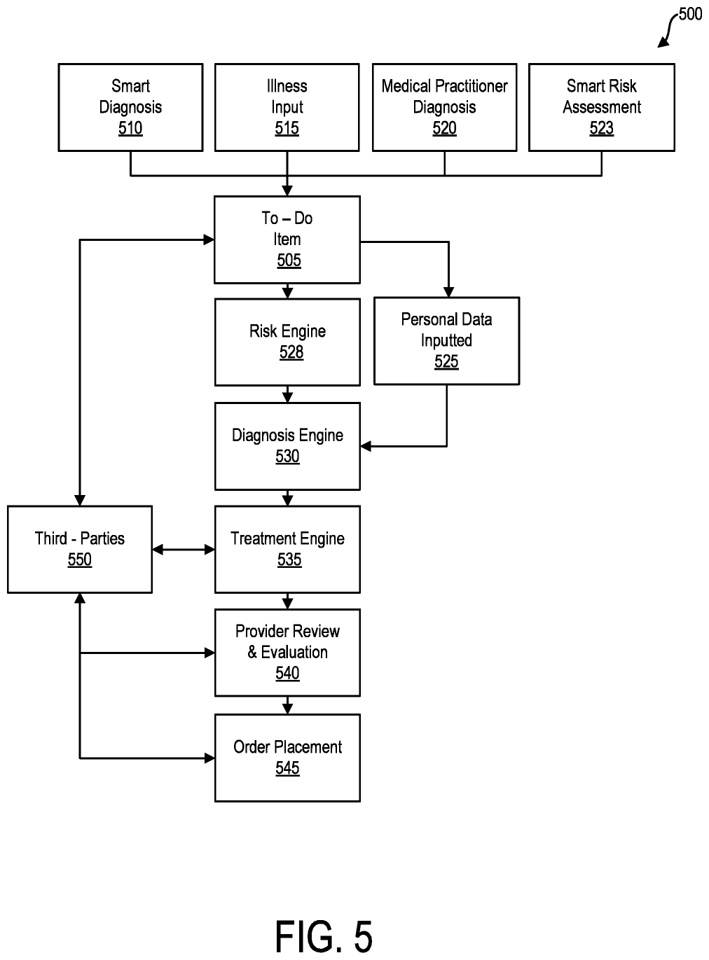

FIG. 5 illustrates an example flowchart 500 for the creation of To-Do Items by a Personal Health Manager system, in accordance with certain implementations described herein. A To-Do Item 505 may be defined as a process created by the PHM that relates to the patient's health and requires execution and completion by the PHM and/or patient, medical practitioner, or a third-party. Examples of To-Do Items 505 are transmitting treatment data to a patient, requesting a medical device, and requesting additional information from the patient or medical practitioner before or after diagnosis of a disease, such as through an action card or via a chatbot.

Generally speaking, the PHM treats all incoming data related to a patient—whether it is received from a To-Do Item, the patient, a third party, or from anywhere else—in much the same way. That is, when the PHM receives the data, it creates a new To-Do Item. The PHM screens the data to determine if there is an existing health concern for the patient, if the data is trending towards a medical concern, or if the PHM can optimize the patient's behavior. Any of this can create an additional To-Do Item. The PHM then updates the Risk Engine for the patient to determine if the data, when combined with other data, leads to any opportunities for treatment, prevention, or optimization. This can also create an additional To-Do Item. The PHM then runs the data through the Diagnostic Engine to determine whether a diagnosis can be generated. The PHM then takes the diagnosis and puts it through the Treatment Engine. This may provide treatment options for the patient, which creates additional To-Do Items. For example, for an existing cough, the PHM may generate actions items that leads to actions such as having an x-ray taken, being prescribed antibiotics, or receiving a referral to labs Following this, the medical practitioner may approve of the results and/or input additional data for the creation of other To-Do Items. However, the approval of or review by a medical practitioner is not essential or even necessary. Rather, the PHM may be sufficiently capable of addressing some medical needs of a patient that approval by a medical practitioner is not required. If treatment options have been generated, the Orders Engine may execute a treatment plan. For example, the patient may receive a referral for an x-ray, a prescription is sent to the pharmacy, or a referral is sent to a lab. The PHM may then follow up on the main condition and on some of the orders. This process repeats itself for all new data received or if additional To-Do Items are created. This process is cyclical and continuously ongoing.

In general terms, as shown in FIG. 5, the PHM may create a To-Do Item 505 through its internal components or processes, such as a smart diagnosis 510 via the Diagnosis Engine, a user inputting data related to a self-diagnosed illness 515, a medical practitioner diagnosis 520, or a smart risk assessment 523 via the Risk Engine. Once the To-Do Item 505 is created, the PHM may determine whether additional data is needed from the patient. If the PHM determines additional data is required from the patient before continuing with the execution and completion of the To-Do Item 505, the PHM may electronically notify the patient. The PHM may receive data subsequently inputted 525 by the patient, such as through the patient inputting the data into the patient interface. Once the PHM has determined it has sufficient data to proceed it will run the To-Do Item 505 through its Risk Engine 528, Diagnosis Engine 530, and Treatment Engine 535 to generate a new risk analysis, diagnosis, and/or treatment options based on the existing data the PHM has already received. The PHM may render and display the proposed risk, diagnosis, and/or treatment on the patient or medical practitioner's interface. The medical practitioner, such as a nurse or a doctor, may be required to review 540, evaluate, amend if necessary, and approve of final decisions. The PHM may receive additional data from the medical practitioner and generate an order placement 545 via the Orders Engine based on the input and approval provided by the medical practitioner.

In one embodiment of the example flowchart 500, the PHM may have previously gathered data relevant to a patient and diagnosed 510 the patient with a medical condition such as bronchitis. The PHM may create a To-Do Item 505 for the treatment of the patient's bronchitis. The PHM may receive data 525 from the patient as to whether it has previously been diagnosed with bronchitis and then run all data through the Risk Engine 528, Diagnosis Engine 530, and Treatment Engine 535. The Risk Engine 528 may evaluate the data and run it through risk calculators to determine whether the patient's data exceed any thresholds or are cause for the development of health concerns. The Diagnosis Engine 530 may evaluate the data, compare it to data on drchrono™ and the patient's medical history data residing on the medical practitioner's server, and diagnosis the patient with acute bronchitis, as opposed to chronic bronchitis. Based on this diagnosis, the PHM may search the database of third-parties 550 to determine what medication is required to treat acute bronchitis and which third-party providers 550 are capable of providing the medication within the timeframe designated by the Treatment Engine 530. The PHM may render and display the acute bronchitis diagnosis and corresponding treatment options to the medical practitioner 540 who may either change the diagnosis or treatment and/or approve. Based on the medical practitioner's determination and data input, the PHM may then generate orders 545 such as placing requests for the purchase and delivery of the medication via a third-party 550 or informing the patient, via the patient interface, that the patient must go see a specialist.

In another embodiment of the example flowchart 500, the PHM may receive notification via the chatbot on the patient interface that the patient believes he is ill 515. The PHM may create a To-Do Item 505 to diagnose the patient with an illness. The PHM may create and render tailored questions on the patient interface, such as via the chatbot, to obtain additional data necessary to diagnose the patient. The PHM may then run this data through the Smart Engine 530 and the Treatment Engine 535 and diagnose the patient with influenza. The PHM may provide this data to the medical provider 540 who will communicate with a third-party urgent care clinic 550 to determine treatment options that are in close proximity to the patient. The PHM may generate an order placement 545 to the third-party urgent care clinic 550 for the patient to visit a medical practitioner there.

Similarly, in another embodiment, the example flowchart 500 may be initiated by the PHM receiving a diagnosis from a medical practitioner 520 after visiting with the patient in the medical practitioner's office. The medical practitioner may input data into the PHM to create a To-Do Item 505 for the treatment of the patient's diagnosed condition. The PHM may then carry out some or all of the steps in the example flowchart 500 to treat the patient based on the medical practitioner's diagnosis 520. Or the example flowchart 500 may be initiated by the PHM receiving a risk assessment 523 from its Risk Engine.

It is noted, for the purposes of this disclosure, that the creation of To-Do Items 505 is not restricted to either the smart diagnosis 510, Illness Input 515, medical practitioner diagnosis 520, or smart risk assessment 523. The PHM may create To-Do Items 505 in a variety of ways. For example, after the PHM has run data through the Treatment Engine 535 it may, in addition to requiring provider review and evaluation 540, also create a new, additional To-Do Item 505. Or, by continuously monitoring incoming user data or monitoring internal or external databases for updated health data—such as through device apps or Internet-of-things devices—the PHM may create To-Do Items 505 via action cards on a recurring basis to engage with the patient, with the purpose of obtaining additional data for generating the remaining life expectancy of the patient and for the future diagnosis and treatment of the patient. The PHM may create To-Do Items 505, via the Diagnosis or Risk Engines, from symptoms reported via the chatbot, from data received following the patient's execution of action cards, from receiving updated data from an external database, or even from a casual chat conversation between a medical practitioner and the patient, wherein the Diagnosis or Risk Engine detect potential illnesses from the patient's text data.

In another example, the PHM breaks down long-term health goals into generated daily actions (i.e. To-Do Items and/or action cards) that impact the patient's longevity. In so doing, the PHM carries out a process of monitoring and continually engaging with the client for the purpose of managing the patient's healthcare. Currently, healthcare is carried out via a doctor, with data dumped on the patient all at one time with a lot of things happen in between each visit. The PHM is able to take this doctor-patient interaction, granulate it, and administer the ongoing personal healthcare management of the patient; turning daily patient interaction into to-do items that the patient can then engage with via the PHM to optimize one's health, longevity, and outlook—all with very little human involvement. Similarly, when a medical practitioner is involved, the PHM can generate and recommend options for the diagnosis and treatment of the patient. And the PHM can connect patients and medical practitioners with third-parties capable of providing relevant services and products for the healthcare management of the patient.

And, the PHM may generate a database that third parties can use to offer, provide, advertise, or sell their services and products to patients and medical practitioners. The PHM may enable third parties to strategically target and directly communicate with patients and medical practitioners via interfaces based on the action cards, to-do items, diagnosis, risk analyses, and treatment options generated by the PHM.

As shown in FIG. 5, third-party connectivity 550 may occur at any point in the example flowchart 500. For example, the Treatment Engine 535, medical practitioner interface, and Orders Engine may communicate with third parties. Or, To-Do Items 505 may transmit data directly to third parties 550.

The PHM may comprise a currency system—such as the use of virtual coins, cryptocurrency, or PHM-specific currency—through which users may receive compensation for engaging with the PHM (i.e., engaging with action cards or with other users). The currency system may also enable users to provide currency in exchange for accessing additional capabilities of the PHM that can further benefit the user's health.

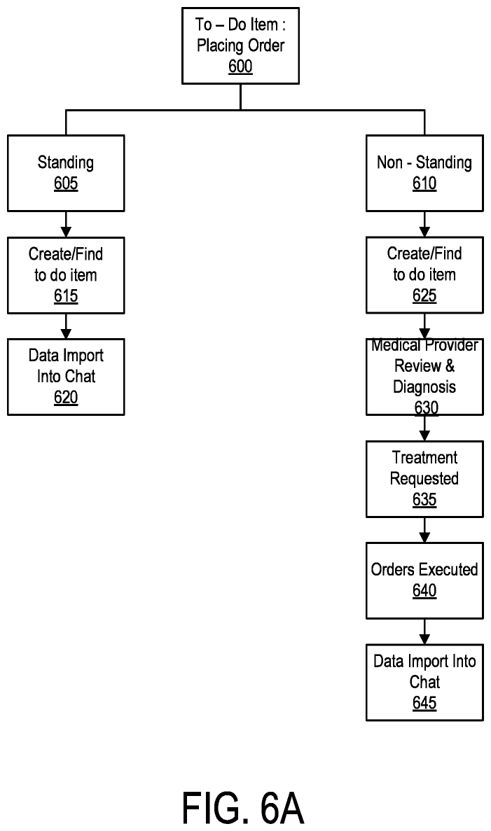

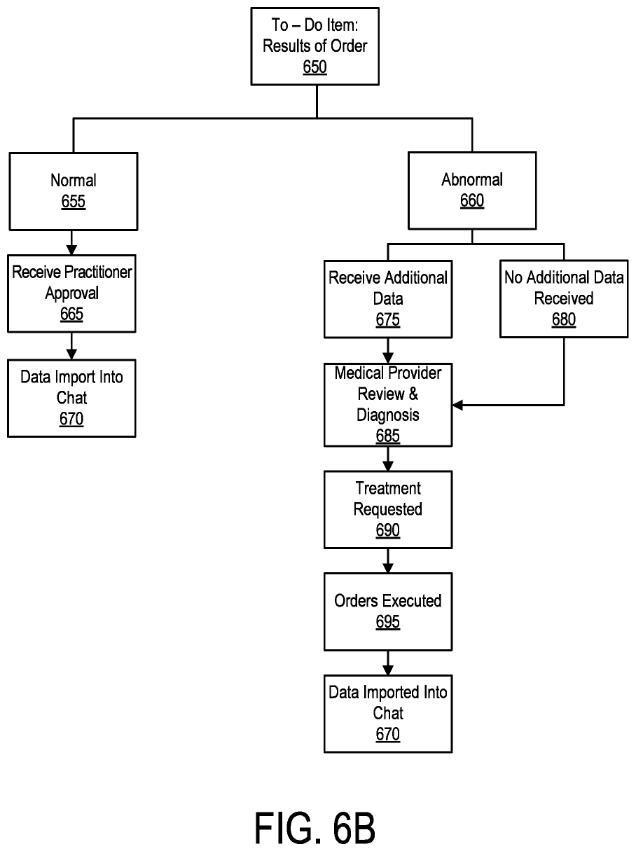

FIG. 6A illustrates example processes for the execution of a To-Do Item by a Personal Health Manager system, in accordance with certain implementations described herein. As shown in FIG. 6A, a To-Do Item may be placing an order 600, which may be the result of the PHM generating a diagnosis, risk analysis, or treatment option. The PHM may categorize To-Do Items such as order placements 600 into Standing 605 and Non-Standing 610 items. Standing 605 Items may be defined as To-Do Items 600 that do not require a medical practitioner's review or approval. As a result, Standing 605 Items may be automatically carried out by the PHM with minimal human supervision or input. In one embodiment, the PHM may designate a Standing 605 Item and automatically execute the placing an order 600, such as requesting lab results, and import the resulting data into the patient interface, such as via the chatbot 620.

Non-Standing 610 Items may be defined as To-Do Items 600 that require a medical practitioner's review and approval. For example, it may only require the review of a nurse, or it may require review and approval by a doctor. The PHM may designate a To-Do Item 600 as a Non-Standing 610 Item, create any required to-do items 625 to execute the Non-Standing 610 Item, and generate and render the results on the medical practitioner's interface for review and approval 630. Following confirmation by the medical practitioner, the PHM may generate any requested treatment actions 635 from a third-party, generate order placement 640 for any necessary orders, and import this data 645 into the patient's interface for the patient to be updated on the status of his health treatment.

FIG. 6B illustrates example processes for the execution of a To-Do Item by a Personal Health Manager system, in accordance with certain implementations described herein. As shown in FIG. 6B, a To-Do Item may be analyzing the Results of an Order 650. The PHM may categorize Results of Order 650 as Normal 655 or Abnormal 660. The PHM may run Results 650 through its Diagnosis Engine for determination of whether additional data is needed or to determine whether it requires additional review and/or input from either the patient or medical practitioner. The PHM may categorize as Normal 655 those Results 650 which require only medical practitioner approval 665. Following receipt of medical practitioner approval 665, the PHM may import the relevant data into the patient interface 670. The PHM may categorize as Abnormal 660 those Results 650 which require additional data and/or review. Following the Abnormal 660 categorization, the PHM request and receive additional data 675 or proceed without additional data 680. The PHM may receive medical practitioner review and approval of the diagnosis 685, and then generate any requested treatment actions 690 from a third-party, generate order placement 695 for any necessary orders, and import this data 670 into the patient's interface for the patient to be updated on the status of his health treatment.

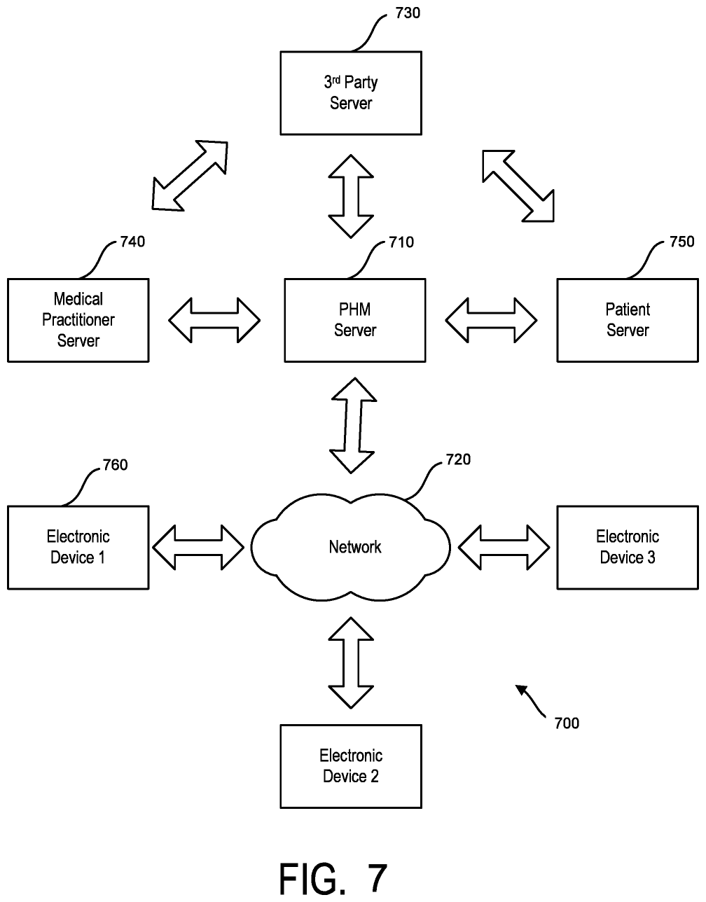

FIG. 7 is a functional block diagram generally illustrating an embodiment of a network system 700 for a Personal Health Manager system. A network system 700, as shown in FIG. 7, may comprise a personal health management server 710 (“PHM server”) accessible over a local area network or a wide area network 720, such as the Internet. The PHM server 710 may enable third-party servers 730, medical practitioner servers 740, patient servers 750, and electronic devices 760 to connect to the PHM server 710. In accordance with the preferred embodiment, the PHM server 710 is remotely accessible by a number of user computing devices 760, including for example, laptops, smartphones, computers, tablets, and other computing devices that are able to access the local area network or a wide area network where the PHM server 710 resides. In normal operation, each user electronic device 760 connects with the PHM server 710 to interact with the PHM system.

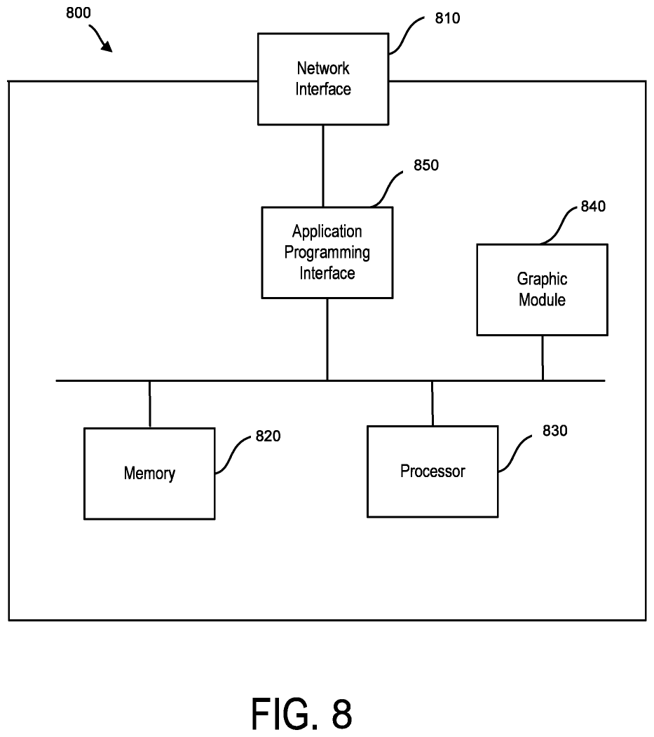

FIG. 8 is a functional block diagram generally illustrating an embodiment of an electronic device system 800 for a Personal Health Manager system. The electronic device 800 may be coupled to a server via a network interface 810. The electronic device 800 generally comprises a memory 820, a processor 830, a graphics module 840, and an application programming interface 850. The electronic device 800 is not limited to any particular configuration or system.

Other embodiments may include combinations and sub-combinations of features described or shown in the several figures, including for example, embodiments that are equivalent to providing or applying a feature in a different order than in a described embodiment, extracting an individual feature from one embodiment and inserting such feature into another embodiment; removing one or more features from an embodiment; or both removing one or more features from an embodiment and adding one or more features extracted from one or more other embodiments, while providing the advantages of the features incorporated in such combinations and sub-combinations. As used in this paragraph, “feature” or “features” can refer to structures and/or functions of an apparatus, article of manufacture or system, and/or the steps, acts, or modalities of a method.

References throughout this specification to “one embodiment,” “an embodiment,” “an example embodiment,” etc., indicate that the embodiment described may include a particular feature, structure, or characteristic, but every embodiment may not necessarily include that particular feature, structure, or characteristic. Moreover, such phrases are not necessarily referring to the same embodiment. Further, when a particular feature, structure, or characteristic is described in connection with one embodiment, it will be within the knowledge of one skilled in the art to affect such feature, structure, or characteristic in connection with other embodiments whether or not explicitly described.

Unless the context clearly indicates otherwise (1) the word “and” indicates the conjunctive; (2) the word “or” indicates the disjunctive; (3) when the article is phrased in the disjunctive, followed by the words “or both,” both the conjunctive and disjunctive are intended; and (4) the word “and” or “or” between the last two items in a series applies to the entire series.

Where a group is expressed using the term “one or more” followed by a plural noun, any further use of that noun to refer to one or more members of the group shall indicate both the singular and the plural form of the noun. For example, a group expressed as having “one or more members” followed by a reference to “the members” of the group shall mean “the member” if there is only one member of the group.

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

What is claimed is:

1. A method in a computer-implemented system, comprising:
receiving, by the system, electronic medical data of a first user;
comparing, by the system, the electronic medical data to a plurality of healthcare data in one or more databases;
identifying, by the system, at least one medical consideration in the electronic medical data upon the comparison of the electronic medical data to the plurality of healthcare data;
comparing, by the system, the identified medical consideration to a plurality of medical consideration data in one or more databases, wherein the plurality of medical consideration data comprises one or more medical considerations and one or more action items addressing the one or more medical considerations;
identifying, by the system, at least one action item addressing the identified medical consideration;
generating, by the system, at least one functional electronic action item corresponding with the identified action item, wherein the functional electronic action item comprises a required data input functionality; and
transmitting, by the system, the functional electronic action item to one or more servers.

2. The method of claim 1, further comprising:
comparing, by the system, the identified medical consideration to a plurality of treatment data in one or more databases, wherein the plurality of treatment data comprises one or more treatment options related to treating the one or more medical considerations;
identifying, by the system, at least one treatment option for treatment of the identified medical consideration; and
transmitting, by the system, the identified medical consideration and the identified treatment option to the one or more servers.

3. The method of claim 2, further comprising:
receiving from the one or more servers, by the system, an offer of a service or product related to at least one of, the identified medical consideration, the identified action item, the identified treatment option, and combinations thereof.

4. The method of claim 2, wherein the electronic medical data comprises at least one of, data inputted into the required data input functionality, data from the one or more servers, data inputted into the system by the first user, data inputted into the system by a second user, and combinations thereof.

5. The method of claim 1, further comprising:
updating, by the system, the plurality of healthcare data and the plurality of medical consideration data;
comparing, by the system, the electronic medical data to the updated plurality of healthcare data in one or more databases;
identifying, by the system, at least one new medical consideration in the electronic medical data upon the comparison of the electronic medical data to the updated plurality of healthcare data;
comparing, by the system, the identified new medical consideration to the updated plurality of medical consideration data in one or more databases, wherein the updated plurality of medical consideration data comprises one or more updated medical considerations and one or more updated action items addressing the one or more medical considerations;
identifying, by the system, at least one new action item addressing the identified new medical consideration;
generating, by the system, at least one new functional electronic action item corresponding with the identified new action item, wherein the new functional electronic action item comprises a required data input functionality; and
transmitting, by the system, the new functional electronic action item to the one or more servers.

6. The method of claim 2, further comprising:
updating, by the system, the plurality of treatment data;
comparing, by the system, the identified medical consideration to the updated plurality of treatment data in one or more databases, wherein the updated plurality of treatment data comprises one or more updated treatment options related to treating the one or more medical considerations;
identifying, by the system, at least one new treatment option for the treatment of the identified medical consideration; and
transmitting, by the system, the identified new medical consideration and the identified new treatment option to the one or more servers.

7. The method of claim 5, further comprising:
comparing, by the system, the identified new medical consideration to a plurality of treatment data in one or more databases, wherein the plurality of treatment data comprises one or more treatment options related to treating the one or more medical considerations;
identifying, by the system, at least one treatment option for the treatment of the identified new medical consideration; and
transmitting, by the system, the identified new medical consideration and the identified treatment option to the one or more servers.

8. A non-transitory, computer-readable storage medium storing instructions, the instructions comprising one or more instructions that, when executed by one or more processors, cause the one or more processors to:
receive electronic medical data of a first user;
compare the electronic medical data to a plurality of healthcare data in one or more databases;
identify at least one medical consideration in the electronic medical data upon the comparison of the electronic medical data to the plurality of healthcare data;
compare the identified medical consideration to a plurality of medical consideration data in one or more databases, wherein the plurality of medical consideration data comprises one or more medical considerations and one or more action items addressing the one or more medical considerations;
identify at least one action item addressing the identified medical consideration;
generate at least one functional electronic action item corresponding with the identified action item, wherein the functional electronic action item comprises a required data input functionality; and
transmit the functional electronic action item to one or more servers.

9. The non-transitory, computer-readable storage medium of claim 8, further comprising instructions that cause the one or more processors to:
compare the identified medical consideration to a plurality of treatment data in one or more databases, wherein the plurality of treatment data comprises one or more treatment options related to treating the one or more medical considerations;
identify at least one treatment option for treatment of the identified medical consideration; and
transmit the identified medical consideration and the identified treatment option to the one or more servers.

10. The non-transitory, computer-readable storage medium of claim 9, further comprising instructions that cause the one or more processors to:
transmit to the one or more servers at least one of, the identified medical consideration, the identified action item, the identified treatment option, and combinations thereof;
receive from the one or more servers an offer of a service or product related to at least one of, the identified medical consideration, the identified action item, the identified treatment option, and combinations thereof.

11. The non-transitory, computer-readable storage medium of claim 9, wherein the electronic medical data of a first user comprises at least one of, data inputted into the required data input functionality, data from the one or more servers, data inputted into the system by the first user, data inputted into the system by a second user, and combinations thereof.

12. The non-transitory, computer-readable storage medium of claim 8, further comprising instructions that cause the one or more processors to:
update the plurality of healthcare data and the plurality of medical consideration data;
compare the electronic medical data to the updated plurality of healthcare data in one or more databases;
identify at least one new medical consideration in the electronic medical data upon the comparison of the electronic medical data to the updated plurality of healthcare data;
compare the identified new medical consideration to the updated plurality of medical consideration data in one or more databases, wherein the updated plurality of medical consideration data comprises one or more updated medical considerations and one or more updated action items addressing the one or more medical considerations;
identify at least one new action item addressing the identified new medical consideration;
generate at least one new functional electronic action item corresponding with the identified new action item, wherein the new functional electronic action item comprises a required data input functionality; and
transmit the new functional electronic action item to the one or more servers.

13. The non-transitory, computer-readable storage medium of claim 9, further comprising instructions that cause the one or more processors to:
update the plurality of treatment data;
compare the identified medical consideration to the updated plurality of treatment data in one or more databases, wherein the updated plurality of treatment data comprises one or more updated treatment options related to treating the one or more medical considerations;
identify at least one new treatment option for the treatment of the identified medical consideration; and
transmit the identified new medical consideration and the identified new treatment option to the one or more servers.

14. The non-transitory, computer-readable storage medium of claim 12, further comprising instructions that cause the one or more processors to:
compare the identified new medical consideration to a plurality of treatment data in one or more databases, wherein the plurality of treatment data comprises one or more treatment options related to treating the one or more medical considerations;
identify at least one treatment option for the treatment of the identified new medical consideration; and
transmit the identified new medical consideration and the identified treatment option to the one or more servers.

15. A computing device comprising:
a processor; and
a non-transitory computer-readable medium operably coupled to the processor, the computer-readable medium having computer-readable instructions stored thereon that, when executed by the processor, cause the computing device to,
receive electronic medical data of a first user;
compare the electronic medical data to a plurality of healthcare data in one or more databases;
identify at least one medical consideration in the electronic medical data upon the comparison of the electronic medical data to the plurality of healthcare data;
compare the identified medical consideration to a plurality of medical consideration data in one or more databases, wherein the plurality of medical consideration data comprises one or more medical considerations and one or more action items addressing the one or more medical considerations;
identify at least one action item addressing the identified medical consideration;
generate at least one functional electronic action item corresponding with the identified action item, wherein the functional electronic action item comprises a required data input functionality; and
transmit the functional electronic action item to one or more servers.

16. The computing device of claim 15, wherein the computer-readable instructions further cause the computing device to:
compare the identified medical consideration to a plurality of treatment data in one or more databases, wherein the plurality of treatment data comprises one or more treatment options related to treating the one or more medical considerations;
identify at least one treatment option for treatment of the identified medical consideration; and
transmit the identified medical consideration and the identified treatment option to the one or more servers.

17. The computing device of claim 16, wherein the computer-readable instructions further cause the computing device to:
transmit to the one or more servers at least one of, the identified medical consideration, the identified action item, the identified treatment option, and combinations thereof;
receive from the one or more servers an offer of a service or product related to at least one of, the identified medical consideration, the identified action item, the identified treatment option, and combinations thereof.

18. The computing device of claim 16, wherein the electronic medical data of a first user comprises at least one of, data inputted into the required data input functionality, data from the one or more servers, data inputted into the system by the first user, data inputted into the system by a second user, and combinations thereof.

19. The computing device of claim 15, wherein the computer-readable instructions further cause the computing device to:
update the plurality of healthcare data and the plurality of medical consideration data;
compare the electronic medical data to the updated plurality of healthcare data in one or more databases;
identify at least one new medical consideration in the electronic medical data upon the comparison of the electronic medical data to the updated plurality of healthcare data;
compare the identified new medical consideration to the updated plurality of medical consideration data in one or more databases, wherein the updated plurality of medical consideration data comprises one or more updated medical considerations and one or more updated action items addressing the one or more medical considerations;
identify at least one new action item addressing the identified new medical consideration;
generate at least one new functional electronic action item corresponding with the identified new action item, wherein the new functional electronic action item comprises a required data input functionality; and
transmit the new functional electronic action item to the one or more servers.

20. The computing device of claim 16, wherein the computer-readable instructions further cause the computing device to:
update the plurality of treatment data;
compare the identified medical consideration to the updated plurality of treatment data in one or more databases, wherein the updated plurality of treatment data comprises one or more updated treatment options related to treating the one or more medical considerations;
identify at least one new treatment option for the treatment of the identified medical consideration; and
transmit the identified new medical consideration and the identified new treatment option to the one or more servers.
